(19) United States
(12) Patent Application Publication
(10) Pub. No.: US 2010/0144858 A1
(43) Pub. Date: Jun. 10, 2010

(54) Treatment of movement disorders with a metabotropic glutamate 4 receptor positive allosteric modulator

(76) Inventors: P. Jeffrey Conn, Brentwood, TN (US); Anthony G. DiLella, Lansdale, PA (US); Gene G. Kinney, Collegeville, PA (US); Michael J. Marino, Souderton, PA (US); Guy R. Seabrook, Blue Bell, PA (US); David L. Williams, Telford, PA (US)

Correspondence Address: Gerard Bilotto, 80 Winding Ridge Road, White Plains, NY, 10603, US

(21) Appl. No.: 12/592,499

(22) Filed: Nov. 25, 2009

Related U.S. Application Data
(63) Continuation of application No. 10/564,029, filed on Jan. 9, 2006, now abandoned.
(60) Provisional application No. 60/486,691, filed on Jul. 11, 2003.

(51) Int. Cl. A61K 31/352; A61P 25/16

(52) U.S. Cl. 514/456

(57) ABSTRACT

An mGluR4 receptor positive allosteric modulator is useful, alone or in combination with a neuroleptic agent, for treating or preventing movement disorders such as Parkinson's disease, dyskinesia, tardive dyskinesia, drug-induced parkinsonism, postencephalitic parkinsonism, progressive supranuclear palsy, multiple system atrophy, corticobasal degeneration, parkinsonian-ALS dementia complex, basal ganglia calcification, akinesia, akinetic-rigid syndrome, bradykinesia, dystonia, medication-induced parkinsonian, Gilles de la Tourette syndrome, Huntington's disease, tremor, chorea, myoclonus, tick disorder, and dystonia.

This application is a continuation of U.S. application Ser. No. 10/564,029, which is the National stage of international application PCT/US2004/021776 filed Jul. 7, 2004, which claims the benefit under 35 U.S.C. §119(e) of U.S. Provisional application Ser. No. 60/486,691, filed Jul. 11, 2003.

### BACKGROUND OF THE INVENTION

The excitatory amino acid L-glutamate (sometimes referred to herein simply as glutamate) through its many receptors mediates most of the neurotransmission within the mammalian central nervous system (CNS). The excitatory amino acids, including glutamate, are of great physiological importance, playing a role in a variety of physiological processes, such as long term potentiation (learning and memory), the development of synaptic plasticity, motor control, respiration, cardiovascular regulation, and sensory perception.

Glutamate acts via at least two distinct classes of receptors. One class is composed of the ionotropic glutamate (iGlu) receptors that as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

The mGluR receptors belong to the Type III G-protein coupled receptor (GPCR) superfamily. This superfamily of GPCR's which includes the calcium-sensing receptors, GABAB receptors and pheromone receptors, are unique in that they are activated by binding of agonists to a large amino-terminus portion of the receptor protein. The mGlu receptors are thought to mediate glutamate's demonstrated ability to modulate intracellular signal transduction pathways. Ozawa, Kamiya and Tsuzuski, Prog. Neurobio., 54, 581 (1998). They have been demonstrated to be localized both pre- and post-synaptically where they can regulate neurotransmitter release, either glutamate or other neurotransmitters, or modify the post-synaptic response of neurotransmitters, respectively.

Diseases of the extrapyramidal motor systems cause either a loss of movement (akinesia) accompanied by an increase n muscle tone (rigidity) or abnormal involuntary movements (dyskinesias) often accompanied by a reduction in muscle tone. The akinetic-rigid syndrome called Parkinsonism, and the dyskinesias represent opposite ends of the spectrum of movement disorders (for review see C. D. Marsden in Oxford Textbook of Medicine, 3^{rd} Edition, Oxford University Press, 1996, vol. 3, pages 3998-4022).

Treatment of akinetic-rigid conditions such as parkinsonism typically involves the use of levodopa, anticholinergics or dopamine agonists. Levodopa is converted into dopamine in the brain by the enzyme dopa decarboxylase. However, this enzyme is also present in the gut wall, liver, kidney and cerebral capillaries, thus the peripheral formation of levodopa metabolites may give rise to side-effects such as nausea, vomiting, cardiac dysrhythmias and postural hypotension. This peripheral decarboxylation is largely prevented by the addition of a selective extracerebral decarboxylase inhibitor, such as carbidopa or benserazide, which themselves do not penetrate the brain. Levodopa combined with carbidopa (SINEMET™) or benserazide (MADOPAR™) is now the treatment of choice when levodopa is indicated. Even then, this combination therapy may be associated with side-effects such as dyskinesias and psychiatric disturbances.

An anticholinergic such as benzhexol or orphenadrine may be used, however, anticholinergics cause peripheral parasympathetic blockade which may cause dry mouth, blurred vision and constipation, and they may also precipitate glaucoma, urinary retention and a toxic confusional state.

Dopamine agonists such as bromocriptine (PARLODEL™), lisuride and pergolide (CELANCE™) act directly on dopamine receptors and have a similar side-effect profile to levodopa.

The dyskinesias, notably tremor, chorea, myoclonus, tics and dystonias, are treated with a variety of pharmacological agents. Thus, for example, tremor may be treated with benzodiazepines such as diazepam; chorea may be treated with diazepam, a phenothiazide or haloperidol, or tetrabenazine; tics may be controlled with neuroleptics such as haloperidol or pimozide; and dystonias tend to be treated with levodopa, benzodiazepines such as diazepam, anticholinergics such as benzhexol, phenothiazines and other neuroleptics such as haloperidol, and tetrabenazine.

Treatment of psychotic disorders with neuroleptic agents, such as haloperidol may be at the expense of a number of side-effects, including extrapyramidal symptoms, acute dystonias, tardive dyskinesias, akathesia, tremor, tachycardia, drowsiness, confusion, postural hypotension, blurring of vision, precipitation of glaucoma, dry mouth, constipation, urinary hesitance and impaired sexual function. There exist patient populations that are resistant to dopamine replacement therapy, as well as populations in whom dyskinesias are inadequately treated with existing antiparkinsonian therapy. Furthermore, some patients may be adversely affected by the extrapyramidal side-effects of neuroleptic drugs.

Thus, existing therapy for movement disorders, especially Parkinson's disease, has centered on replacement of lost dopaminergic tone through the use of direct or indirect dopamine agonists. While these methods are initially successful, most patients experience a dramatic decrease in efficacy and the development of severe adverse side effects within 5 years of beginning therapy. The mechanism of these adverse effects is not fully understood, however it is clear that they are related to the use of dopamine replacement. In view of the short-comings of existing therapy, there is a need for new, safe and effective treatment for movement disorders.

In accordance with the present invention, agents acting down-stream of the dopamine system as positive allosteric modulators of the mGluR4 receptor restore balance in the basal ganglia motor circuit. The use of a positive allosteric modulator of the mGluR4 receptor bypasses the dopamine system and would provide long lasting palliative benefit without producing the side effects associated with dopamine replacement. In addition to providing palliative relief from the symptoms of movement disorders, this re-normalization of circuit activity results in a decrease in glutamate release in the substantia nigra pars compacta dopamine neurons thereby arresting degeneration of these neurons in movement disorders such as Parkinson's disease.

### SUMMARY OF THE INVENTION

The present invention is directed to the use of a positive allosteric modulator of the mGluR4 receptor, alone or in combination with a neuroleptic agent, for treating, preventing the progression, ameliorating, controlling or reducing the risk of movement disorders such as Parkinson's disease, dyskinesia, tardive dyskinesia, drug-induced parkinsonism, postencephalitic parkinsonism, progressive supranuclear palsy, multiple system atrophy, corticobasal degeneration, parkinsonian-ALS dementia complex, basal ganglia calcification, akinesia, akinetic-rigid syndrome, bradykinesia, dystonia, medication-induced parkinsonian, Gilles de la Tourette syndrome, Huntington's disease, tremor, chorea, myoclonus, tick disorder, and dystonia.

### DESCRIPTION OF THE INVENTION

The present invention is directed to the use of a positive allosteric modulator of the mGluR4 receptor, alone or in combination with other neuroleptic agents, for treating, preventing the progression, ameliorating, controlling or reducing the risk of movement disorders such as Parkinson's disease, dyskinesia, tardive dyskinesia, drug-induced parkinsonism, postencephalitic parkinsonism, progressive supranuclear palsy, multiple system atrophy, corticobasal degeneration, parkinsonian-ALS dementia complex, basal ganglia calcification, akinesia, akinetic-rigid syndrome, bradykinesia, dystonia, medication-induced parkinsonian, Gilles de la Tourette syndrome, Huntington's disease, tremor, chorea, myoclonus, tick disorder, and dystonia.

An embodiment of the present invention is directed to a method for treating, preventing the progression, ameliorating, controlling or reducing the risk of a movement disorder in a patient in need thereof that comprises administering to the patient a therapeutically effective amount of a positive allosteric modulator of the mGluR4 receptor or a pharmaceutically acceptable salt thereof

An embodiment of the present invention is directed to a method for treating, preventing the progression, ameliorating, controlling or reducing the risk of Parkinson's disease in a patient in need thereof that comprises administering to the patient a therapeutically effective amount of an mGluR4 receptor positive allosteric modulator or a pharmaceutically acceptable salt thereof.

An embodiment of the present invention is directed to a method for treating, preventing the progression, ameliorating, controlling or reducing the risk of a dyskinesia in a patient in need thereof who is non-responsive to neuroleptic agents or for whom neuroleptic agents are contraindicated, that comprises administering to the patient a therapeutically effective amount of an mGluR4 receptor positive allosteric modulator or a pharmaceutically acceptable salt thereof.

By the term “mGluR4 receptor positive allosteric modulator” is meant any exogenously administered compound or agent that directly or indirectly augments the activity of the mGluR4 receptor in the presence or in the absence of the endogenous ligand (such as glutamate) in an animal, in particular, a human. The term “mGluR4 receptor positive allosteric modulator” includes a compound that is an “mGluR4 receptor allosteric potentiator” or an “mGluR4 receptor allosteric agonist”, as well as a compound that has mixed activity as both an “mGluR4 receptor allosteric potentiator” and an “mGluR4 receptor allosteric agonist”.

By the term “mGluR4 receptor allosteric potentiator” is meant any exogenously administered compound or agent that directly or indirectly augments the response produced by the endogenous ligand (such as glutamate) when it binds to the orthosteric site of the mGluR4 receptor in an animal, in particular, a human. The mGluR4 receptor allosteric potentiator binds to a site other than the orthosteric site (an allosteric site) and positively augments the response of the receptor to an agonist. Because it does not induce desensitization of the receptor, activity of a compound as an mGluR4 receptor allosteric potentiator provides advantages over the use of a pure mGluR4 receptor allosteric agonist. Such advantages may include, for example, increased safety margin, higher tolerability, diminished potential for abuse, and reduced toxicity.

By the term “mGluR4 receptor allosteric agonist” is meant any exogenously administered compound or agent that directly augments the activity of the mGluR4 receptor in the absence of the endogenous ligand (such as glutamate) in an animal, in particular, a human. The mGluR4 receptor allosteric agonist binds to the orthosteric glutamate site of the mGluR4 receptor and directly influences the orthosteric site of the mGluR4 receptor. Because it does not require the presence of the endogenous ligand, activity of a compound as an mGluR4 receptor allosteric agonist provides advantages over the use of a pure mGluR4 receptor allosteric potentiator, such as more rapid onset of action.

In a preferred embodiment of the present invention, the compound that is an mGluR4 receptor positive allosteric modulator possesses balanced activity as an mGluR4 receptor allosteric potentiator and as an mGluR4 receptor allosteric agonist. In an alternately preferred embodiment of the present invention, combination therapy with a compound that is an mGluR4 receptor allosteric potentiator and with a compound that is an mGluR4 receptor allosteric agonist may be employed.

In an embodiment of the present invention the mGluR4 receptor positive allosteric modulator is a positive allosteric modulator of the human mGluR4 receptor.

In an embodiment of the present invention the mGluR4 receptor positive allosteric modulator possesses a selectivity for the mGluR4 receptor relative to each of the other mGluR receptors of at least 3 fold as measured by the ratio of EC₅₀ for the mGluR4 receptor to the EC₅₀ for each of the other mGluR receptors. In another embodiment of the present invention the mGluR4 receptor positive allosteric modulator possesses a selectivity for the mGluR4 receptor relative to other mGluR receptors of at least 10 fold as measured by the ratio of EC₅₀ for the mGluR4 receptor to the EC₅₀ for other mGluR receptors. In another embodiment of the present invention the mGluR4 receptor positive allosteric modulator possesses a selectivity for the mGluR4 receptor relative to the other mGluR receptors of at least 30 fold as measured by the ratio of EC₅₀ for the mGluR4 receptor to the EC₅₀ for the other mGluR receptors. In another embodiment of the present invention the mGluR4 receptor positive allosteric modulator possesses a selectivity for the mGluR4 receptor relative to the other mGluR receptors of at least 100 fold as measured by the ratio of EC₅₀ for the mGluR4 receptor to the EC₅₀ for the other mGluR receptors. In another embodiment of the present invention the mGluR4 receptor positive allosteric modulator possesses a selectivity for the mGluR4 receptor relative to the other mGluR receptors of at least 300 fold as measured by the ratio of EC₅₀ for the mGluR4 receptor to the EC₅₀ for the other mGluR receptors.

In an embodiment of the present invention the mGluR4 receptor positive allosteric modulator possesses an EC₅₀ for binding to the mGluR4 receptor of 1 uM or less as evaluated by the FLIPR assay. In another embodiment of the present invention the mGluR4 receptor positive allosteric modulator possesses an EC₅₀ for binding to the mGluR4 receptor of 300 nM or less as evaluated by the FLIPR assay. In another embodiment of the present invention the mGluR4 receptor positive allosteric modulator possesses an EC₅₀ for binding to the mGluR4 receptor of 100 nM or less as evaluated by the FLIPR assay. In another embodiment of the present invention the mGluR4 receptor positive allosteric modulator possesses an EC₅₀ for binding to the mGluR4 receptor of 30 nM or less as evaluated by the FLIPR assay. In another embodiment of the present invention the mGluR4 receptor positive allosteric modulator possesses an EC₅₀ for binding to the mGluR4 receptor of 10 nM or less as evaluated by the FLIPR assay. In another embodiment of the present invention the mGluR4 receptor positive allosteric modulator possesses an EC₅₀, for binding to the mGluR4 receptor of 3 nM or less as evaluated by the FLIPR assay. In another embodiment of the present invention the mGluR4 receptor positive allosteric modulator possesses an EC₅₀ for binding to the mGluR4 receptor of 1 nM or less as evaluated by the FLIPR assay.

In an embodiment of the present invention the mGluR4 receptor positive allosteric modulator is an orally active mGluR4 receptor positive allosteric modulator. In an embodiment of the present invention the mGluR4 receptor positive allosteric modulator is orally administered. In another embodiment of the present invention the mGluR4 receptor positive allosteric modulator is a non-peptidyl mGluR4 receptor positive allosteric modulator.

The mGluR4 receptor positive allosteric modulator may be peptidyl or non-peptidyl in nature, however, the use of a non-peptidyl mGluR4 receptor positive allosteric modulator is preferred. In addition, for convenience the use of an orally active mGluR4 receptor positive allosteric modulator is preferred. Similarly, for convenience the use of a once-a-day medicament is preferred.

In an embodiment of the present invention the mGluR4 receptor positive allosteric modulator is a CNS-penetrant mGluR4 receptor positive allosteric modulator and is able to enter the brain and/or central nervous system with sufficient concentration to have a therapeutic effect. In a further embodiment of the present invention the CNS-penetrant mGluR4 receptor positive allosteric modulator is a compound that exhibits sufficient concentration in the brain and/or central nervous system to have therapeutic efficacy upon oral administration.

An embodiment of the present invention is directed to use of the compound N-phenyl-7-(hydroxylimino)cyclopropa[b]chromen-1a-carboxamide (PHCCC) (Annoura, H., Fukunaga, A., Uesugi, M., Tatsouka, T. & Horikawa, Y. (1996) Bioorg. Med. Chem. Lett. 6, 763-766) which has been identified by the inventors as a potentiator of human and rat mGluR4. The inventors have found that PHCCC does not itself exhibit mGluR4 agonist activity. In contrast, the closely related analogue 7-(hydroxylimino)cyclopropa[b]chromen-1a-carboxamide ethyl ester (CPCCOEt) (Annoura, H., Fukunaga, A., Uesugi, M., Tatsouka, T. & Horikawa, Y. (1996) Bioorg. Med. Chem. Lett. 6, 763-766) had no mGluR4 potentiator activity. Characterization of PHCCC revealed that it does not potentiate or activate any other mGluR subtype but acts as an antagonist of some of the mGluRs. In brain slice electrophysiological studies of the rat striato-pallidal synapse, PHCCC was found to potentiate the effect of the mGluR4 agonist L-AP4 in inhibiting transmission. Finally, PHCCC was found to overcome inhibition of movement observed in a dopamine-depletion rat model of Parkinson's disease. These studies support the use of an mGluR4 receptor positive allosteric modulator alone or in combination with other neuroleptic agents, for treating, preventing the progression, ameliorating, controlling or reducing the risk of movement disorders in accordance with the present invention.

Althought the mGluR4 receptor positive allosteric modulator is useful alone for movement disorders, it will be appreciated that a combination of a conventional antiparkinsonian drug with an mGluR4 receptor positive allosteric modulator may provide an enhanced effect in the treatment of akinetic-rigid disorders such as parkinsonism. Such a combination may enable a lower dose of the antiparkinsonian agent to be used without compromising the efficacy of the antiparkinsonian agent, thereby minimising the risk of adverse side-effects.

An embodiment of the present invention is directed to a method for treating, controlling, ameliorating or reducing the risk of an akinetic-rigid disorder in a patient in need therof, that comprises administering to the patient a therapeutically effective amount of an mGluR4 receptor positive allosteric modulator or a pharmaceutically acceptable salt thereof and an amount of an antiparkinsonian agent, such that together they give effective relief.

An embodiment of the present invention is directed to a method for treating, controlling, ameliorating or reducing the risk of a dyskinesia in a patient in need therof, that comprises administering to the patient a therapeutically effective amount of an mGluR4 receptor positive allosteric modulator or a pharmaceutically acceptable salt thereof and an amount of a neuroleptic agent, such that together they give effective relief.

It will be further appreciated that a combination of a conventional neuroleptic drug with mGluR4 receptor positive allosteric modulator or a pharmaceutically acceptable salt thereof may provide an enhanced effect in the treatment of dyskinesias. Such a combination may enable a lower dose of the neuroleptic agent to be used without compromising the efficacy of the neuroleptic agent, thereby minimising the risk of adverse side-effects. A yet further advantage of such a combination is that, due to the action of mGluR4 receptor positive allosteric modulator, adverse side-effects caused by the neuroleptic agent such as acute dystonias, dyskinesias, akathesia and tremor may be reduced or prevented.

The present invention also provides a method for the treatment or prevention of dyskinesias, which method comprises administration to a patient in need of such treatment of an amount of mGluR4 receptor positive allosteric modulator or a pharmaceutically acceptable salt thereof and an amount of a neuroleptic agent, such that together they give effective relief.

As used herein, the term “movement disorders” includes akinesias and akinetic-rigid syndromes, dyskinesias and medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor). Examples of “akinetic-rigid syndromes” include Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification. Examples of “dyskinesias” include tremor (including rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics),and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia).

Another “movement disorder” which may be treated according to the present invention is Gilles de la Tourette's syndrome, and the symptoms thereof.

The term “therapeutically effective amount” shall mean that amount of a drug or pharmaceutical agent that will elicit the biological or medical response of a tissue, system, animal or human that is being sought by a researcher or clinician.

Accordingly, the present invention includes within its scope the use of an mGluR4 receptor positive allosteric modulator, alone or in combination with other agents, for the subject indications in a mammal The preferred mammal for purposes of this invention is human.

The subject treated in the present methods is generally a mammal, preferably a human, male or female. In the present invention, it is preferred that the subject mammal is a human. Although the present invention is applicable both old and young people, in certain aspects such as cognition enhancement it would find greater application in elderly people. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

This particular application of an mGluR4 receptor positive allosteric modulator provides unexpected benefit relative to the administration of other agents for the subject indications. For example, the mGluR4 receptor positive allosteric modulator may exhibit a rapid onset of action and a reduced side-effect profile relative to conventional agents used for the treatment of extrapyramidal movement disorders and other types of movement disorders (e.g. idiopathic Parkinson's disease, secondary Parkinson's disease, Huntingdon's disease, dystonia, chorea, tics, myoclonus and athetosis).

For use in medicine, the salts of the compounds employed in this invention refer to non-toxic “pharmaceutically acceptable salts.” Other salts may, however, be useful in the preparation of the compounds according to the invention or of their pharmaceutically acceptable salts. Salts encompassed within the term “pharmaceutically acceptable salts” refer to non-toxic salts of the compounds of this invention which are generally prepared by reacting the free base with a suitable organic or inorganic acid. Representative salts include the following: Acetate, Benzenesulfonate, Benzoate, Bicarbonate, Bisulfate, Bitartrate, Borate, Bromide, Calcium, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Subacetate, Succinate, Sulfate, Sulfonate, Tannate, Tartrate, Teoclate, Tosylate, Triethiodide and Valerate. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The mGluR4 receptor positive allosteric modulator as employed in the present invention, may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers.

An mGluR4 receptor positive allosteric modulator may be used alone or in combination with other neruoleptic agents or with other compounds which are known to be beneficial in the subject indications. An mGluR4 receptor positive allosteric modulator and the other agent may be co-administered, either in concomitant therapy or in a fixed combination. For example, an mGluR4 receptor positive allosteric modulator may be administered in conjunction with other compounds which are known in the art for the subject indications.

It will be appreciated that when using a combination of the present invention, the mGluR4 receptor positive allosteric modulator and the antiparkinsonian or neuroleptic agent may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, the antiparkinsonian or neuroleptic agent may be administered as a tablet and then, within a reasonable period of time, an mGluR4 receptor positive allosteric modulator may be administered either as an oral dosage form such as a tablet or a fast-dissolving oral dosage form. By a “fast-dissolving oral formulation” is meant, an oral delivery form which when placed on the tongue of a patient, dissolves within about 10 seconds.

In accordance with the present invention, an mGluR4 receptor positive allosteric modulator is useful alone or in combination with other antiparkinsonian agents for treating, controlling, ameliorating or reducing the risk of a movement disorder.

Suitable antiparkinsonian agents of use in combination with the mGluR4 receptor positive allosteric modulator include for example levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

An mGluR4 receptor positive allosteric modulator or a pharmaceutically acceptable salt thereof, may be administered in combination with a compound selected from the group consisting of: acetophenazine, alentemol, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, risperidone, sulpiride, tetrabenazine, trihexyphenidyl, thioridazine, thiothixene and trifluoperazine.

Suitable neuroleptic agents of use in combination with the mGluR4 receptor positive allosteric modulator or a pharmaceutically acceptable salt thereof include the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the mGluR4 receptor positive allosteric modulator may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form.

The present invention includes within its scope a pharmaceutical composition for the subject indications comprising, as an active ingredient, an mGluR4 receptor positive allosteric modulator in association with a pharmaceutical carrier or diluent. Optionally, the active ingredient of the pharmaceutical compositions can comprise another agent in addition to an mGluR4 receptor positive allosteric modulator to minimize the side effects or with other pharmaceutically active materials wherein the combination enhances efficacy and minimizes side effects.

The present invention is further directed to a method for the manufacture of a medicament for the subject indications in humans comprising combining a compound that is an mGluR4 receptor positive allosteric modulator with a pharmaceutical carrier or diluent.

It will be known to those skilled in the art that there are numerous compounds now being used for movement disorders. Combinations of these therapeutic agents some of which have also been mentioned herein with an mGluR4 receptor positive allosteric modulator will bring additional, complementary, and often synergistic properties to enhance the desirable properties of these various therapeutic agents. In these combinations, an mGluR4 receptor positive allosteric modulator and the therapeutic agents may be independently present in dose ranges from one one-hundredth to one times the dose levels which are effective when these compounds and secretagogues are used singly.

To illustrate these combinations, an mGluR4 receptor positive allosteric modulator effective clinically at a given daily dose range may be effectively combined, at levels which are equal or less than the daily dose range, with such compounds at the indicated per day dose range. Typically, the individual daily dosages for these combinations may range from about one-fifth of the minimally recommended clinical dosages to the maximum recommended levels for the entities when they are given singly. It will be readily apparent to one skilled in the art that an mGluR4 receptor positive allosteric modulator may be employed with other agents for the purposes of the present invention.

Naturally, these dose ranges may be adjusted on a unit basis as necessary to permit divided daily dosage and, as noted above, the dose will vary depending on the nature and severity of the disease, weight of patient, special diets and other factors.

These combinations may be formulated into pharmaceutical compositions as known in the art and as discussed below. An mGluR4 receptor positive allosteric modulator may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents. When the unit dosage form is a capsule, it may contain, in addition to materials of the above type, a liquid carrier such as fatty oil. Various other materials may be present as coatings or to otherwise modify the physical form of the dosage unit. Tablets and pills can additionally be prepared with enteric coatings and tablets may be coated with shellac, sugar or both.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring, and perfuming agents. A syrup or elixir may contain the active compound, sucrose as a sweetening agent, methyl and propyl parabens as preservatives, a dye and a flavoring such as cherry or orange flavor.

Preparations according to this invention for parenteral administration include sterile aqueous or non-aqueous solutions, suspensions, or emulsions. Sterile compositions for injection may be formulated according to conventional pharmaceutical practice by dissolving or suspending the active substance in a vehicle such as water for injection, a naturally occurring vegetable oil like sesame oil, coconut oil, peanut oil, cottonseed oil, etc., or a synthetic fatty vehicle like ethyl oleate or the like. Buffers, preservatives, antioxidants and the like may be incorporated as required. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use. Compositions for rectal or vaginal administration may be suppositories which may contain, in addition to the active substance, excipients such as cocoa butter or a suppository wax. Compositions for nasal or sublingual administration are also prepared with standard excipients well known in the art.

It will be appreciated that the amount of the mGluR4 receptor positive allosteric modulator will vary not only with the compositions selected but also with the route of administration, the nature of the condition being treated, and the age and condition of the patient, and will ultimately be at the discretion of the patient's physician or pharmacist.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 10 mg/kg. of body weight daily are administered to the patient, e.g., humans and elderly humans. The dosage range will generally be about 0.5 mg to 1.0 g. per patient per day which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day. Specific dosages for administration include 10 mg, 30 mg and 60 mg.

Pharmaceutical compositions of the present invention may be provided in a solid dosage formulation preferably comprising about 0.5 mg to 500 mg active ingredient, more preferably comprising about 1 mg to 250 mg active ingredient. The pharmaceutical composition is preferably provided in a solid dosage formulation comprising about 1 mg, 5 mg, 10 mg, 25 mg, 50 mg, 100 mg, 200 mg or 250 mg active ingredient.

A minimum dosage level for the antiparkinsonian agent will vary depending upon the choice of agent, but is typically about 0.05 mg per day for the most potent compounds or about 20 mg per day for less potent compounds. A maximum dosage level for the antiparkinsonian agent is typically 30 mg per day for the most potent compounds or 500 mg per day for less potent compounds. The compounds are administered one to three times daily, preferably once or twice a day, and especially once a day.

A minimum dosage level for the neuroleptic agent will vary depending upon the choice of agent, but is typically about 0.5 mg per day for the most potent compounds or about 20 mg per day for less potent compounds. A maximum dosage level for the neuroleptic agent is typically 30 mg per day for the most potent compounds or 200 mg per day for less potent compounds. The compounds are administered one to three times daily, preferably once or twice a day, and especially once a day.

The following examples are provided so that the invention might be more fully understood. These examples are illustrative only and should not be construed as limiting the invention in any way.

Chemicals: PHCCC, CPCCOEt, L-AP4, 6-cyano-7-nitroquinoxaline-2,3-dione (CNQX), D-(−)-2-amino-5-phosphopentanoic acid (D-AP5) (2S)-3-[[1S)-1-(3,4-dichlorophenyl)ethyl]amino-2-hydroxypropyl]phenylmethyl)phosphinic acid (CGP 55845) and glutamate were all purchased from Tocris-Cookson (Ellisville USA).
Cell lines: Cell lines expressing mGluR1b, 2, 4, 5, 7 and 8 were developed that were compatible with Ca2+ sensitive fluorescence assays. Cells expressing mGluR2, mGluR4, mGluR7 and mGluR8 were coexpressed with G_{α16}, G_{αqi5}, G_{α15} and G_{α15}, respectively. Cells were evaluated using a fluorometric imaging plate reader (FLIPR, Molecular Devices, Sunnyvale, Calif.), to measure their ability to mobilize Ca²⁺ in response to appropriate agonists (i.e., glutamate and L-AP4).

### Fluorometric Imaging Plate Reader (FLIPR):

CHO or HEK cells expressing mGluR receptors (mGluR CHO or HEK cells) were plated (50,000-70,000 cells/well) in clear-bottomed, poly-D-lysine-coated plates (Becton-Dickinson) in glutamate/glutamine-free medium. The plated cells were grown overnight at 37° C. in the presence of 6% CO₂. The following day, the cells were washed with 3×100 μl assay buffer (Hanks Balanced Salt Solution containing 20 mM HEPES, 2.5 mM probenecid, and 0.1% bovine serum albumin) using a Skatron Embla cell washer. The cells were incubated with 1 μM Fluo-4AM (Molecular Probes, Eugene, Oreg.) for 1 h at 37° C. and 6% CO₂. The extracellular dye was removed by washing as described above. For potency determination, the cells were pre-incubated in assay buffer with various concentrations of compound for 5 min and then stimulated for 3 min with either an EC₂₀ or EC₅₀ concentration of agonist (i.e. glutamate or L-AP4) for potentiation measurements or antagonist measurements, respectively. Ca²⁺ flux was measured using a FLIPR.

The group I antagonist PHCCC (10 μM) potentiated the response to glutamate (2 TM) 5.3-fold compared to glutamate alone measured in a FLIPR assay measuring increases of intracellular calcium in mGluR4 CHO cells. In the absence of agonist, PHCCC had no effect on the activity of mGluR4. Furthermore, PHCCC (10 μM) did not activate or potentiate responses to any other mGluR subtype examined. However, 10 μM PHCCC partially blocked responses of mGluR1b, mGluR2, mGluR5a, and mGluR8 to glutamate.

PHCCC potentiated the response of human mGluR4 to 50 nM L-AP4 with an EC50 value of 4.1±1.2 μM. Similar values were found using glutamate as the agonist, as well as for rat mGluR4 using either L-AP4 or glutamate as the agonist. L-AP4 concentration-response curves were shifted to the left in the presence of 10 μM PHCCC. EC₅₀ values for L-AP4 activation of human mGluR4 were 484±45 nM (n=3) in the absence of PHCCC and 71.4±2.9 nM (n=3) with 10 μM PHCCC; for rat mGluR4, 832±100 nM (n=3) without PHCCC and 67.6±5.2 nM (n=3) with 10 μM PHCCC. The maximal effect of L-AP4 was increased approximately two-fold in the presence of 10 μM PHCCC, suggesting PHCCC also increases the intrinsic efficacy of agonists.

CPCCOEt was tested in the FLIPR assay for its ability to potentiate mGluR4. CPCCOEt did not have any effect on the response of mGluR4 to agonists at concentrations up to 30 μM, although at higher concentrations it appears to be an mGluR4 antagonist (IC₅₀>100 μM).

Animals: All studies were performed in an AAALAC accredited facility in accordance with all applicable guidelines regarding the care and use of animals. Animals were group housed with access to food and water ad libitum.
Slice Preparation: All experiments were performed on slices from 26 to 30-d-old Sprague Dawley rats (Taconic, Germantown, N.Y.). Animals were killed by decapitation and brains were rapidly removed and submerged in an ice-cold solution containing (in mM): choline chloride 126, KCl 2.5, NaH₂PO₄ 1.2, MgCl₂ 1.3, MgSO₄ 8, glucose 10, and NaHCO₃ 26, equilibrated with 95% O₂/5% CO₂ (11). The brain was glued to the chuck of a vibrating blade microtome (Leics Microsystems, Nussloch GmbH) and parasagittal slices (300 μm thick) were obtained. Slices were immediately transferred to a 500 ml holding chamber containing artificial cerebrospinal fluid (in mM): NaCl 124, KCl 2.5, MgSO₄ 1.3, NaH₂PO₄ 1.0, CaCl₂ 2, glucose 20, and NaHCO₃ 26, equilibrated with 95% O₂/5% CO₂ that was maintained at 32° C. After 20-min at 32° C., the holding chamber was allowed to gradually decrease to room temperature. In all experiments 5 μM glutathione, 500 μM pyruvate, and 250 μM kynurenic acid were included in the choline chloride buffer and in the holding chamber ACSF.
Electrophysiology: Whole-cell patch-clamp recordings were obtained (Marino et al., (2001) J. Neurosci. 21: 7001-7012. During recording, slices were maintained fully submerged on the stage of a 1 ml brain slice chamber at 32° C. and perfused continuously with equilibrated ACSF (2-3 ml/min). Neurons were visualized using a differential interference contrast microscope and an infrared video system. Patch electrodes were pulled from borosilicate glass on a two-stage puller and had resistances in the range of 3-7 MΩ when filled with the following internal solution: (in mM): potassium gluconate 125, NaCl 4, NaH₂PO₄ 6, CaCl₂ 1, MgSO₄ 2, BAPTA-tetrapotassium salt 10, HEPES 10, Mg-ATP 2, Na₂-GTP 0.3, pH=7.4. All recordings were done using HEKA EPC9 patch clamp amplifiers (HEKA Elektronik, Lambrecht/Pfalz, Germany). Inhibitory postsynaptic currents (IPSCs) were evoked in the presence of blockers of AMPA (20 μM CNQX), NMDA (25 μM D-AP-5), and GABA_{B} (100 nM CGP 55845) receptors. Bipolar tungsten stimulation electrodes were placed in the striatum near the border between cortex and striatum. IPSCs were evoked by single pulses that ranged from 30-90 V, 200-400 μsec, delivered once every 30-60 seconds from a holding potential was −50 mV. For hippocampal field recordings a patch electrode filled with ACSF was placed in the dendritic region of CA1 or the dentate gyrus. Field excitatory postsynaptic potentials (fEPSPs) were isolated and characterized (Gereau, R. W. & Conn, P. J. (1995) J. Neurosci. 15, 6879-6889; Macek, T. A., Winder, D. G., Gereau, R. W., Ladd, C. O. & Conn, P. J. (1996) J. Neurophysiol. 76, 3798-3806). Compounds were applied to the bath using a three-way stopcock and were always applied for 10 minutes in order to achieve a plateau concentration.

The compound PHCCC was found to potentiate the effects of a low dose of the group III mGluR agonist L-AP4 on striato-pallidal transmission. Application of 1 μM L-AP4 produced a small but significant inhibition of transmission at the striato-pallidal synapse. Application of vehicle (1% DMSO) or 30 μM (±) PHCCC alone had no effect on striato-pallidal transmission. However, consistent with our findings in recombinant systems, co-application of 30 μM PHCCC and 1 μM L-AP4 produced a marked inhibition (p<0.01 paired t-test n=4). The effect of L-AP4 in the presence of the potentiator was significantly greater than the effect of L-AP4 alone (p<0.05 ANOVA, Fisher's LSD). In order to determine if the selectivity for mGluR4 observed in our recombinant studies was evident in the native slice preparation, we took advantage of two previously characterized synapses in the hippocampus that are known to be modulated by activation of other members of the group III mGluRs. We performed recordings of field excitatory post synaptic potentials (fEPSPs) from the Schaffer collateral-CA1 (SC-CA1) synapse and the lateral perforant path-dentate gyrus (LPP-DG) synapse. Based on the high level of mGluR7 protein and the low potency of L-AP4 at this synapse, this L-AP4-induced decrease in transmission is likely mediated by mGluR7. In addition, it has been suggested that the activation of mGluR8 inhibits transmission at the LPP-DG synapse. We chose submaximal concentrations of L-AP4 that produced a significant decrease in fEPSP slope and looked for potentiation of these effects by PHCCC. Consistent with the results obtained in our recombinant studies, PHCCC produced no significant effect on L-AP4-induced inhibition of transmission at these two synapses. Taken together these findings indicate that PHCCC acts as a selective potentiator of mGluR4 in this native in vitro preparation.

Behavior: Third ventricle cannulated male Sprague-Dawley rats (250-350) were purchased from Taconic Farms (Germantown, N.Y.) with guide cannula implanted such that subsequent placement of an injection cannula allowed for infusion into the third ventricle. These rats were used for intracerebral ventricular (icy) injection of test compounds within one week of arrival to the testing facility. All experiments were carried out during the light cycle (6.00-18.00).

### Induction and Measurement of Akinesia:

Rats were injected with reserpine (5 mg/kg sc, dissolved in 1% acetic acid) and kept in their home cages for 1.5-2 hr post-injection. Activity was measured by placing rats in photocell activity cages (Hamilton-Kinder, Inc., Poway, Calif.) equipped with 16×16 infrared beams. Following a 30 min baseline period, rats were given a single icy injection (0.5 μl/min) of either PHCCC (Tocris, 75 nmol/2.5 μl in vehicle), CPCCOEt (Tocris, 75 nmol/2.5 μl in vehicle) or vehicle control (2.5 μl 40% DMSO in 0.85% NaCl). Five min following the injection of test compound or vehicle, motor activity was recorded for an additional 30 min for each rat. Motor activity (cumulative beam breaks/30-min period) was recorded both pre- and post-drug treatment for each rat. Changes in motor activity were analyzed using a repeated-measures two-factor analysis of variance, where treatment (pre- versus post-drug; within factor) and drug (PHCCC, CPCCOEt, and vehicle; between factor) values were used for each rat. Post hoc comparisons were performed using the Bonferroni test. Statistical significance was achieved when p<0.05. Data are expressed as mean +/− one SEM.

Allosteric Potentiation of the mGluR4 Receptor Produces an Antiparkinsonian Effect in a Dopamine Depletion Akinesia Model:

The ability of PHCCC to reverse motor deficits was tested in a reserpine-induced akinesia rodent model of Parkinson's disease. PHCCC produced a significant increase in locomotor activity whereas vehicle or CPCCOEt treatment had no effect under the same conditions. This observation was confirmed by the finding of significant main effects for test drug, treatment (pre- versus post-drug), and the interaction between drug and treatment (drug effect, F(2,9)=6.53, p<0.05; treatment effect, F(1,9)=30.53, p<0.001; drug x treatment interaction, F(2,9)=12.39, p<0.01). Prior to administering test compounds (pre-drug), the level of reserpine-induced movement deficits for rats randomly assigned to vehicle, PHCCC, and CPCCOEt treatment groups were similar (F(2,9)=1.01, p=0.40). Post hoc analysis revealed that PHCCC, but not vehicle or CPCCOEt, demonstrated significantly greater activity following treatment (p<0.001). Taken together, these findings indicate that PHCCC is a positive allosteric modulator of mGluR4 in both recombinant and native systems. The in vivo antiparkinsonian actions of PHCCC support the present invention that activation of mGluR4 represents a therapeutic approach for the treatment of movement disorders, such as Parkinson's disease.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method of treatment of Parkinson's disease in a patient in need-thereof that comprises administering to the patient a therapeutically effective amount of an mGluR4 receptor positive allosteric modulator or a pharmaceutically acceptable salt thereof consisting of a selectivity for the mGluR4 receptor relative to other mGluR receptors of at least three fold as measured by the ratio of EC₅₀.

2. The method of claim 1, wherein the mGluR4 receptor modulator is N-phenyl-(hydroxylimino)cyclo-propa[b]chromen-1a-carboxamide.

3. The method of claim 1, wherein the mGluR receptor positive allosteric modulator or a pharmaceutically acceptable salt thereof is administered in combination with levodopa.

4. The method of claim 1, wherein the mGluR receptor positive allosteric modulator or a pharmaceutically acceptable salt thereof is administered in combination with levodopa and a selective extracerebral decarboxylase inhibitor.

5. The method of claim 1, wherein the mGluR receptor positive allosteric modulator or a pharmaceutically acceptable salt thereof is administered in combination with a cholinergic agonist and an mGluR receptor allosteric agonist.

6. A method of treatment of Parkinson's disease in a patient in need-thereof that comprises administering to the patient a therapeutically effective amount of an mGluR4 receptor positive allosteric modulator or pharmaceutically acceptable salt thereof having a binding of at least 1 nM for the mGluR4 receptor as measured by EC₅₀ and as evaluated by the Fluorometric Imaging Plate Reader (FLIPR).

7. The method of claim 6, wherein the mGluR receptor positive allosteric modulator or pharmaceutically acceptable salt thereof is administered in combination with an anticholinergic agent.

8. The method of claim 6, wherein the mGluR receptor positive allosteric modulator or pharmaceutically acceptable salt thereof is administered in combination with a dopamine agonist.

9. The method of claim 6, wherein the mGluR receptor positive allosteric modulator or pharmaceutically acceptable salt thereof is administered in combination with neuroleptic agents.

10. A method of positive allosteric modulation of a human mGluR4 receptor having a preferential selectivity of at least three fold as measured by the ratio of EC₅₀ of the mGluR4 to other mGluR receptors and a binding for the mGluR4 receptor relative to the other mGluR receptors as measured by EC₅₀ and evaluated by FLIPR of less than 1 nM.

11. The method of claim 10, wherein the mGluR receptor positive allosteric modulator or pharmaceutically acceptable salt thereof is administered in combination with an mGluR receptor allosteric agonist.

12. The method of claim 10, wherein the mGluR receptor positive allosteric modulator or pharmaceutically acceptable salt thereof is administered in combination with a COMT inhibitor.

13. The method of claim 10, wherein the mGluR receptor positive allosteric modulator or pharmaceutically acceptable salt thereof is administered in combination with an antiparkinsonian agent and a pharmaceutically acceptable carrier or excipient.
